(19) United States
(12) Patent Application Publication
(10) Pub. No.: US 2019/0059804 A1
(43) Pub. Date: Feb. 28, 2019

(54) Systems and Apparatuses for Determining Biomechanical Properties of Tissue and Related Methods

(71) Applicant: The Board of Regents of The University of Texas System, Austin, TX (US)

(72) Inventors: Panayiotis S. Shiakolas, Irving, TX (US); Michael Abraham, Plano, TX (US); Philippe E. Zimmern, Dallas, TX (US); Christopher E. Abrego, Bedford, TX (US)

(21) Appl. No.: 16/109,496

(22) Filed: Aug. 22, 2018

Related U.S. Application Data
(60) Provisional application No. 62/548,647, filed on Aug. 22, 2017.

(51) Int. Cl. A61B 5/00; F16H 19/00

(52) U.S. Cl. CPC A61B 5/4337; F16H 19/005; A61B 2562/0247; A61B 5/7475; F16H 2019/008; A61B 5/0053

(57) ABSTRACT

Some of the present apparatuses for determining biomechanical properties of tissue include an elongated body having a proximal end and a distal end, the elongated body including: a shaft having a longitudinal axis; and an actuatable member pivotally coupled to the shaft, the actuatable member comprising: a base; and a tip configured to be coupled to the base, the tip having a sensor that is configured to collect data indicative of a force exerted on the actuatable member; wherein the actuatable member is movable relative to the shaft between a first position and a second position in which the tip is farther from the longitudinal axis of the shaft than when the actuatable member is in the first position.

### CROSS-REFERENCE TO RELATED APPLICATIONS

This application claims priority to U.S. Provisional Application No. 62/548,647, the entire contents of which is specifically incorporated by reference herein without disclaimer.

### BACKGROUND

### 1. Field of Invention

The present invention relates generally to apparatuses, systems, and methods for use in medical procedures in which one or more biomechanical properties of a tissue are determined, such as, for example, viscoelastic properties of tissue.

### 2. Description of Related Art

Pelvic organ prolapse (POP) is a condition affecting many women each year. POP is a result of the weakening of support for pelvic organs such as the uterus, bladder, or colon. Consequently, female pelvic organs prolapse (i.e., descend) and push against the vaginal walls. In some cases, severe POP may result in the protrusion of the pelvic organs or vaginal walls outside of the body.

An American female has an 11.1% lifetime risk of undergoing an operation for pelvic organ prolapse at the age of 80 (A. L. Olsen, V. J. Smith, J. O. Bergstrom, J. C. Colling and A. L. Clark, “Epidemiology of surgically managed pelvic organ prolapse and urinary incontinence,” Obstetrics & Gynecology, vol. 89, pp. 501-506, 1997), and 75% of patients aged 18 to 83 presenting for a routine gynecological examination show some level of loss of vaginal or uterine support (S. Swift, P. Woodman, A. O. Boyle, M. Kahn, M. Valley, D. Bland, W. Wang and J. Schaffer, “Pelvic Organ Support Study (POSST): The distribution, clinical definition, and epidemiologic condition of pelvic organ support defects,” American Journal of Obstetrics and Gynecology, vol. 192, pp. 795-806, 2005). The condition is especially more common in older age groups (50 years and older). Patients of POP experience pain, urinary incontinence, difficulty of intercourse, impediment of motion, and a general decline in positive body image.

The risk of prolapse can be attributed to multiple factors such as aging, childbirth, obesity, smoking, chronic straining, etc. However, the development of POP is thought to be brought on by a change in the biomechanical properties of the pelvic muscles and connective tissues (C. J. Chuong, M. Ma, P. Zimmern and R. C. Eberhart, “European Journal of Obstetrics & Gynecology and Reproductive Biology Viscoelastic properties measurement of the prolapsed anterior vaginal wall : a patient-directed methodology,” European Journal of Obstetrics and Gynecology, vol. 173, pp. 106-112, 2014). The anterior vaginal wall (AVW) is one of the regions that is most affected by this condition. Physicians in general, urologists in particular, assess this change in AVW biomechanical properties using a traditional pelvic exam, similar to the digital rectal exam performed to diagnose prostate enlargement in males. However, such tests are inherently subjective and qualitative in nature, with no measurable or comparable data across patients and physicians.

### SUMMARY

Some embodiments of the present apparatuses for determining biomechanical properties of tissue comprise an elongated body having a proximal end and a distal end, the elongated body including: a shaft having a longitudinal axis; and an actuatable member pivotally coupled to the shaft, the actuatable member comprising: a base; and a tip configured to be coupled to the base, the tip having a sensor that is configured to collect data indicative of a force exerted on the actuatable member; wherein the actuatable member is movable relative to the shaft between a first position and a second position in which the tip is farther from the longitudinal axis of the shaft than when the actuatable member is in the first position.

In some embodiments, the shaft includes a stop configured to limit pivotal movement of the actuatable member relative to the shaft. In some embodiments, the stop limits pivotal movement of the actuatable member to approximately 50 degrees from the first position.

In some embodiments, the tip is configured to be detachably coupled to the base. In some embodiments, the tip comprises a sensor cover configured to transfer the force exerted on the actuatable member to the sensor.

In some embodiments, the body includes gradations configured to indicate a distance relative to the sensor.

Some embodiments of the present apparatuses comprise an actuator configured to move the actuatable member between the first position and the second position. In some embodiments, a motor coupled to the actuatable member via a belt such that the motor moves the actuatable member by moving the belt.

Some embodiments of the present systems for determining biomechanical properties of tissue comprise an apparatus coupled to the frame, wherein the apparatus includes: an elongated body having a proximal end and a distal end, the elongated body including: a shaft having a longitudinal axis; and an actuatable member pivotally coupled to the shaft, the actuatable member comprising: a base; and a tip configured to be coupled to the base, the tip having a sensor that is configured to collect data indicative of a force exerted on the actuatable member; wherein the actuatable member is movable relative to the shaft between a first position and a second position in which the tip is farther from the longitudinal axis of the shaft than when the actuatable member is in the first position; an actuator, the actuator configured to move the actuatable member between the first position and the second position; a graphics user interface (GUI) configured to receive one or more test parameters; and a controller configured to control the actuator based on the one or more test parameters and receive, from the sensor, data indicative of the force exerted on the actuatable member.

In some embodiments, the one or more test parameters comprise one or both of the following: a time duration of movement of the actuatable member from the first position to the second position; a time duration of holding the actuatable member in the second position; and a time duration of movement of the actuatable member from the second position to the first position.

In some embodiments, the tip is configured to be detachably coupled to the base.

Some embodiments of the present methods for determining biomechanical properties of tissue comprise inserting a distal end of an apparatus into an insertion site on a patient, wherein the apparatus comprises: an elongated body having a proximal end and a distal end, the elongated body including: a shaft having a longitudinal axis; and an actuatable member pivotally coupled to the shaft, wherein: the actuatable member comprises a sensor that is configured to collect data indicative of a force exerted on the actuatable member, the actuatable member is movable relative to the shaft between a first position and a second position in which a tip of the actuatable member is farther from the longitudinal axis of the shaft than when the actuatable member is in the first position; actuating the actuatable member between the first position to the second position; sensing, via the sensor, data indicative of the force exerted on the actuatable member while actuating the actuatable member between the first position and the second position.

Some embodiments of the present methods comprise holding the actuatable member in the second position for approximately one second to approximately ten seconds.

In some embodiments, the data indicative of the force exerted on the actuatable member is sensed while holding the actuatable member in the second position.

In some embodiments, the actuatable member is actuated from the first position to the second position in approximately one-half seconds to approximately three seconds. In some embodiments, the actuatable member is actuated from the second position to the first position in approximately one-half seconds to approximately three seconds.

Some embodiments of the present methods comprise curve-fitting a viscoelastic model to the data sensed by the sensor.

The term “coupled” is defined as connected, although not necessarily directly, and not necessarily mechanically; two items that are “coupled” may be unitary with each other. The terms “a” and “an” are defined as one or more unless this disclosure explicitly requires otherwise. The term “substantially” is defined as largely but not necessarily wholly what is specified (and includes what is specified; e.g., substantially 90 degrees includes 90 degrees and substantially parallel includes parallel), as understood by a person of ordinary skill in the art. In any disclosed embodiment, the term “substantially” may be substituted with “within [a percentage] of” what is specified, where the percentage includes 0.1, 1, 5, and 10 percent.

Further, a device or system that is configured in a certain way is configured in at least that way, but it can also be configured in other ways than those specifically described.

The terms “comprise” (and any form of comprise, such as “comprises” and “comprising”), “have” (and any form of have, such as “has” and “having”), and “include” (and any form of include, such as “includes” and “including”) are open-ended linking verbs. As a result, an apparatus that “comprises,” “has,” or “includes” one or more elements possesses those one or more elements, but is not limited to possessing only those one or more elements. Likewise, a method that “comprises,” “has,” or “includes,” one or more steps possesses those one or more steps, but is not limited to possessing only those one or more steps.

Any embodiment of any of the apparatuses, systems, and methods can consist of or consist essentially of—rather than comprise/have/include—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb.

The feature or features of one embodiment may be applied to other embodiments, even though not described or illustrated, unless expressly prohibited by this disclosure or the nature of the embodiments.

Some details associated with the embodiments are described above and others are described below.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers. In the figures, reference numerals having the same number but different suffixes (e.g., a, b, c, d, or the like) may be used to refer to similar features or features with similar functionality. The figures are drawn to scale (unless otherwise noted), meaning the sizes of the depicted elements are accurate relative to each other for at least the embodiment depicted in the figures.

FIG. 1 is a perspective view of an embodiment of the present apparatuses.

FIGS. 2-7 are top, bottom, first side, second side, third side, and fourth side views, respectively, of the apparatus of FIG. 1.

FIG. 8 is a partial cross section view of the apparatus of FIG. 1, taken along line 8-8 of FIG. 2.

FIG. 9 is a perspective view of an embodiment of an elongated body that may be suitable for use with some embodiments of the present apparatuses.

FIGS. 10-13 are first side, second side, third side, and fourth side views, respectively, of the elongated body of FIG. 9.

FIG. 14 is a magnified view of a portion of FIG. 8, showing an actuatable member that may be suitable for use with some embodiments of the present apparatuses.

FIG. 15 is an exploded view of the actuatable member of FIG. 14.

FIG. 16 is a schematic of an embodiment of the present systems.

FIG. 17 depicts the averaged calibration data points and calibration curve of one embodiment of the present sensors.

FIG. 18 depicts an actuation profile of one embodiment of the present actuators.

FIG. 19 is a graph showing test data of one embodiment of the present apparatuses.

FIGS. 20A and 20B are graphs showing test data of one embodiment of the present apparatuses.

### DETAILED DESCRIPTION OF ILLUSTRATIVE EMBODIMENTS

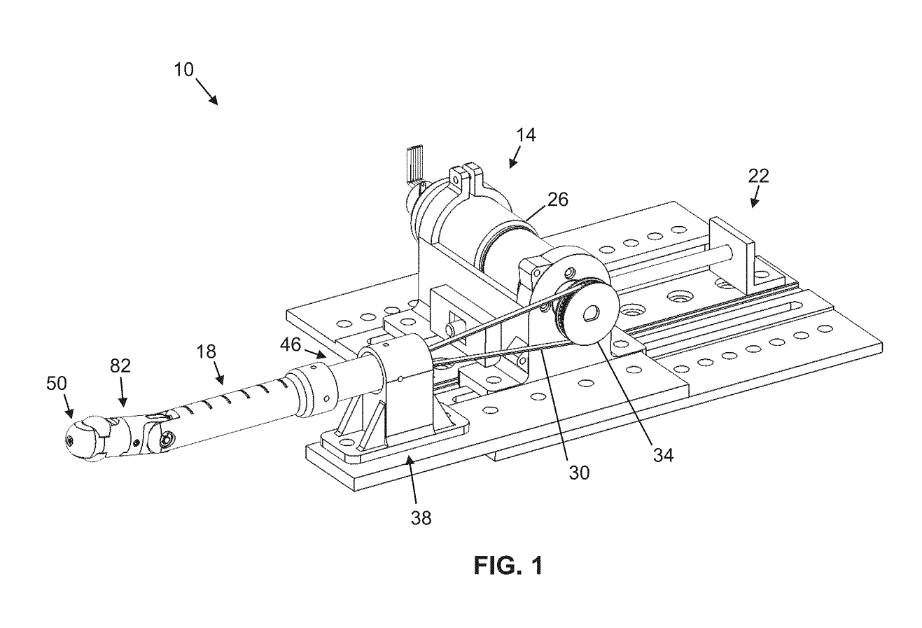

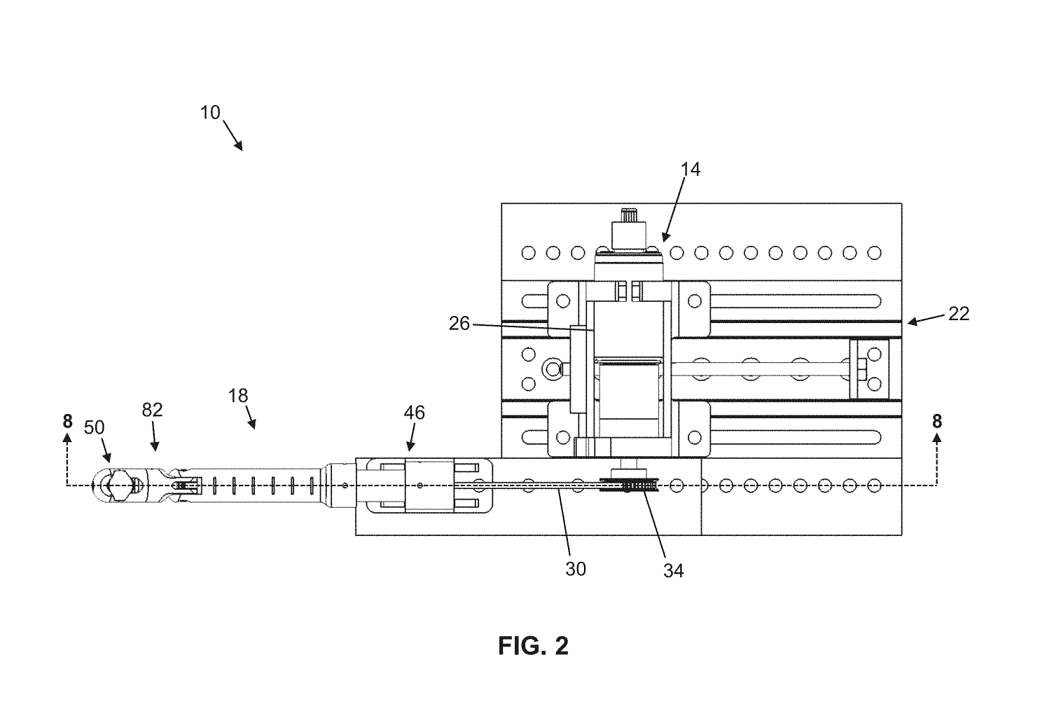

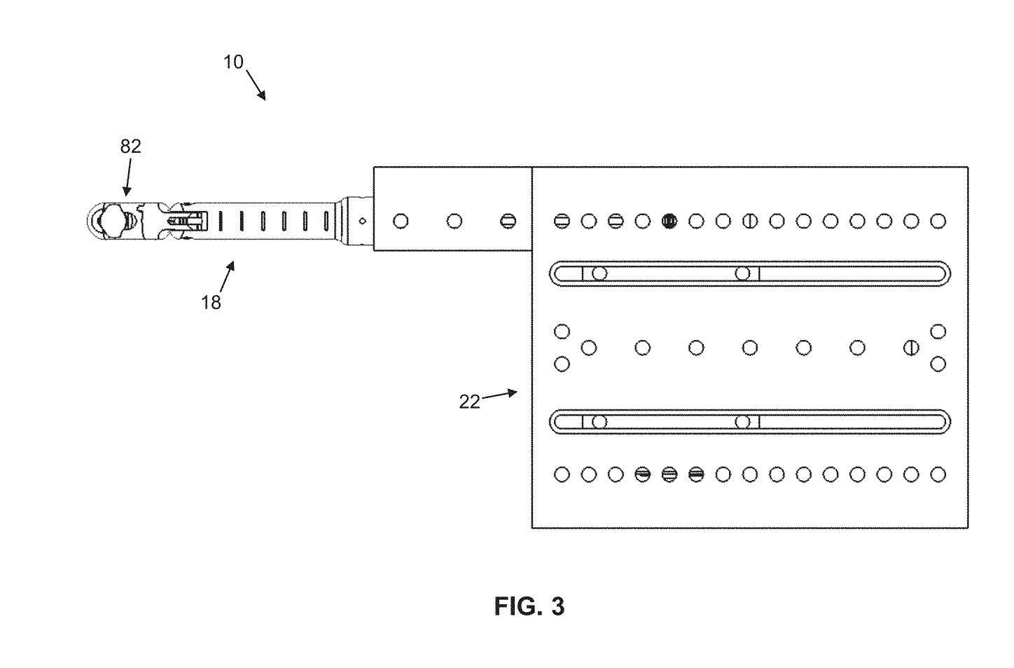

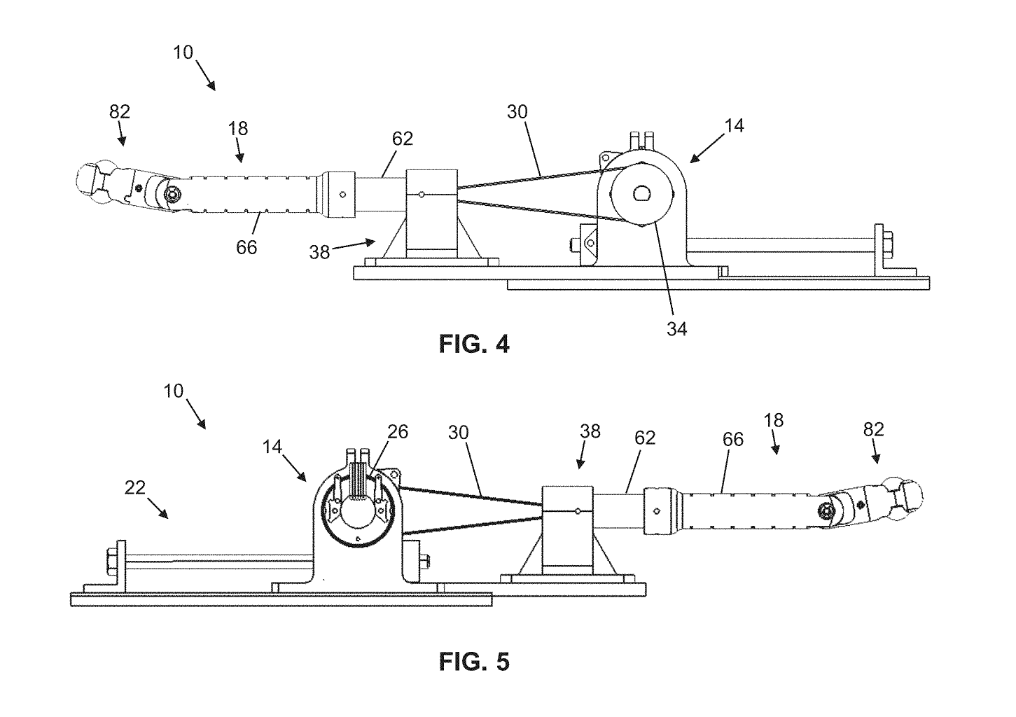

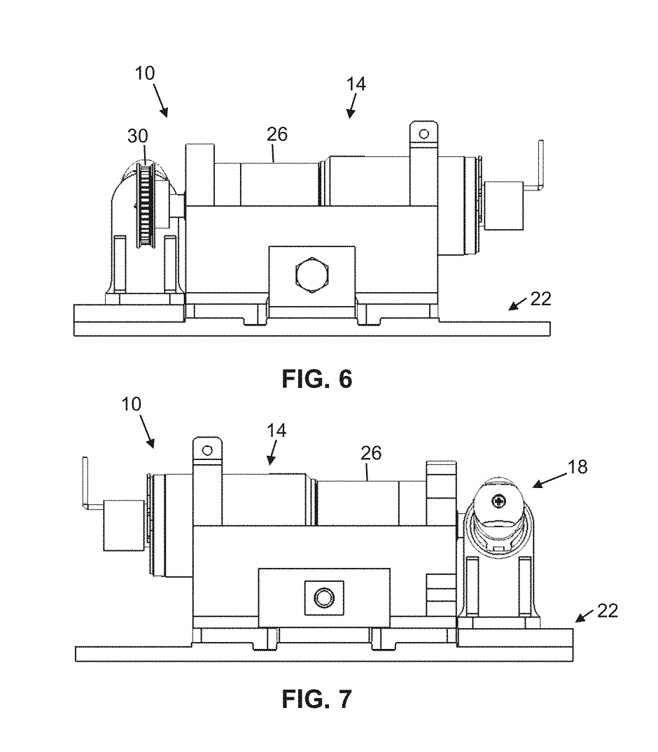

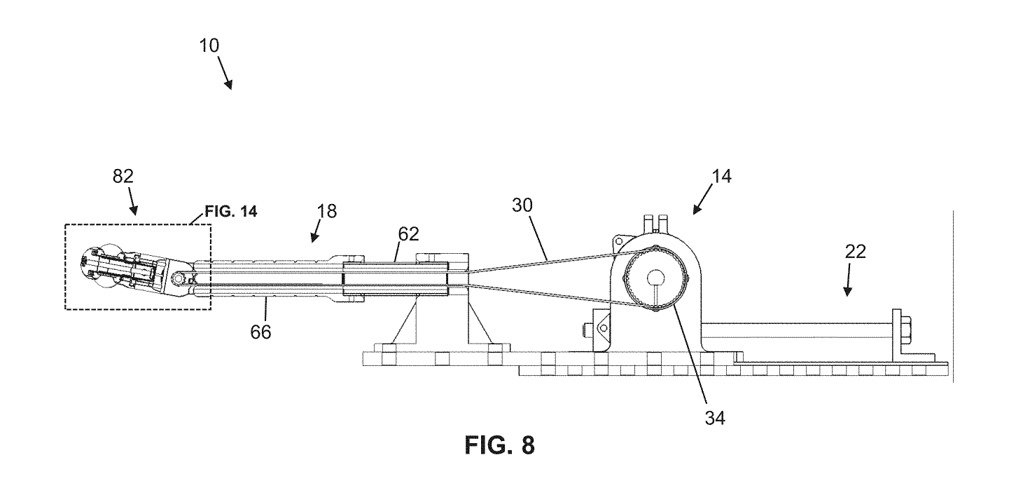

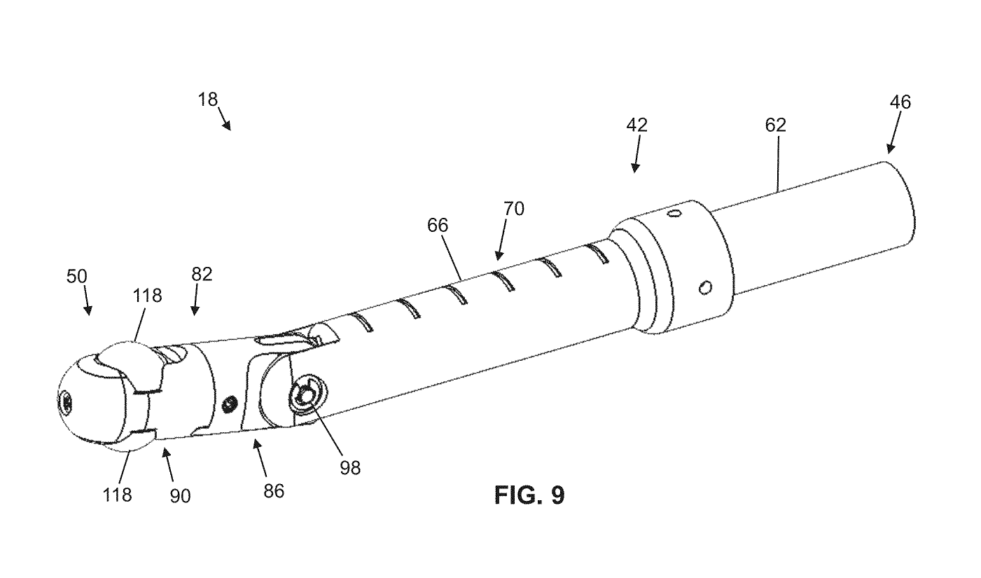

Referring now to the figures, and more particularly to FIGS. 1-8, shown therein and designated by the reference numeral 10 is an embodiment of the present apparatuses for determining biomechanical (e.g., viscoelastic) properties of tissue (e.g., vaginal tissue, anal tissue, skin, and/or the like). More particularly, apparatus 10 can be used to quantitatively determine a stiffness of tissue. In some embodiments, apparatus 10 can be used to quantitatively determine a stiffness of vaginal tissue, such as, for example, tissue of an anterior vaginal wall (AVW).

Apparatus 10 includes an actuator 14 and an elongated body 18. As shown, actuator 14 and body 18 can be configured to be supported by a frame 22. For example, actuator 14 and/or body 18 may be coupled to frame 22 in any suitable position such that the actuator can actuate the body as described herein. In this embodiment, actuator 14 includes a motor 26 and a belt 30 whose movement is controlled by a gear 34 that is coupled to the motor. In this embodiment, actuator 14 includes any suitable motor (e.g., 26) or combination of motors, such as, for example a DC motor, an AC motor, stepper motor, and/or the like. In the depicted embodiment, actuator 14 is configured to move an actuatable member (e.g., 82) of body 18 between a first position and a second position, as described in further detail below.

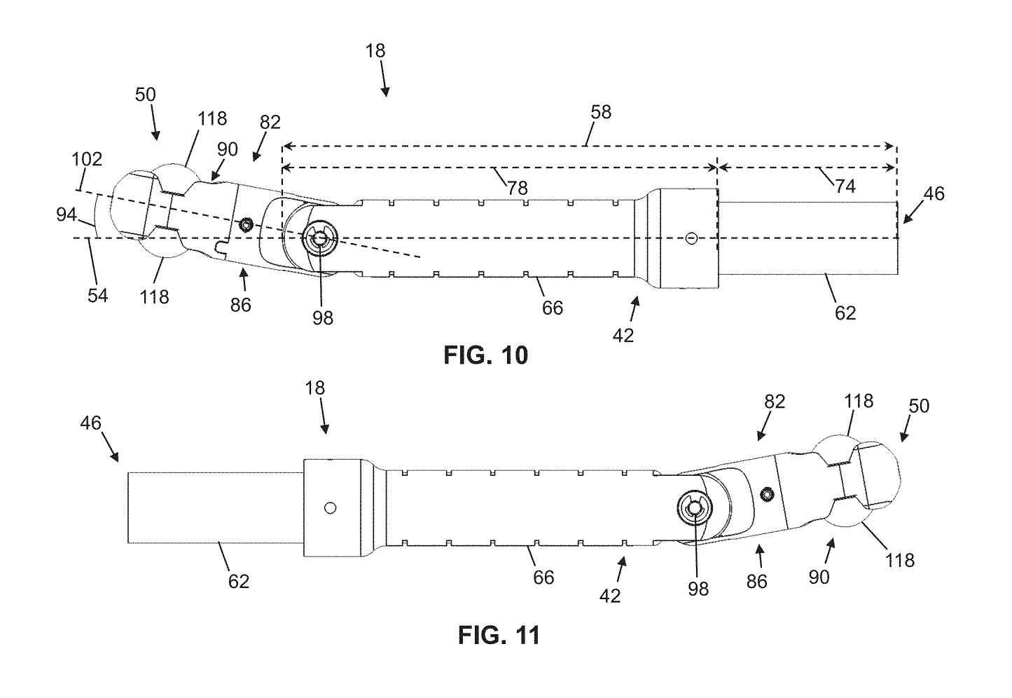

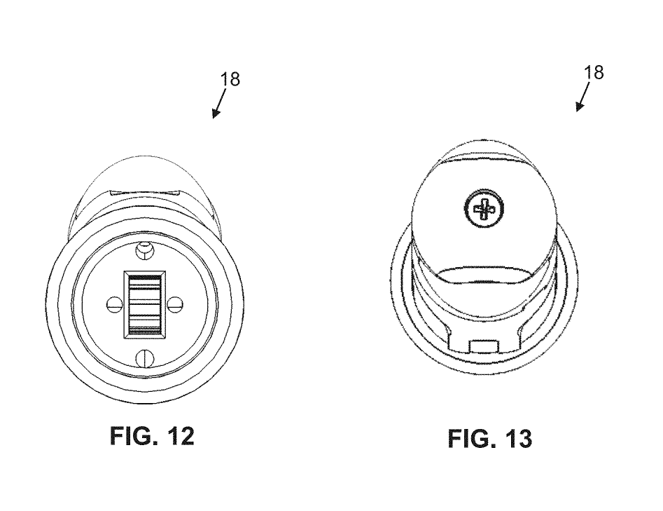

In the depicted embodiment, body 18 is coupled to frame 22 by an anchor member 38. Bodies (e.g., 18) of the present apparatuses may comprise any suitable material, such as, for example, a metal (e.g., stainless steel, aluminum, titanium, and/or the like), plastic, composite material, and/or the like. As shown, body 18 includes a (e.g., rigid) shaft 42 disposed between a proximal end 46 and a distal end 50 of the body. As shown in FIG. 10, shaft 42 includes a longitudinal axis 54. Shaft 42 may comprise any suitable length 58 (e.g., measured along a direction that is substantially parallel relative to longitudinal axis 54) in order to accommodate sufficient insertion of the shaft into an insertion site (e.g., a vagina) of a patient. In this embodiment, shaft 42 includes an extension tube 62 coupled to a penetration tube 66. Extension tube 62 may comprise any suitable cross-sectional shape, such as, for example, circular, elliptical, or otherwise rounded, triangular, square, or otherwise polygonal. At least a portion of a length of extension tube 62 can be hollow. Extension tube 62 may be adjustably coupled to penetration tube 66 such that length 58 of shaft 42 can be adjusted. In some embodiments, a shaft (e.g., 42) includes an extension tube (e.g., 62) that is unitary with a penetration tube (e.g., 66). At least one of frame 22, penetration tube 66, and extension tube 62 may include gradations 70 configured to indicate an insertion depth of shaft 42 in an insertion site of a patient. More particularly, gradations 70 may be configured to indicate a distance (e.g., measured along a direction that is substantially parallel relative to longitudinal axis 54) from a sensor (e.g., 114) of an actuatable member (e.g., 82). Extension tube 62 can comprise a length 74 (e.g., measured along a direction that is substantially parallel relative to longitudinal axis 54) that is any suitable length, such as, for example, approximately any one of, or between approximately any two of, the following: 10, 20, 30, 40, 50, 60, 70, 80, 90, and 100 millimeters (mm). Similarly, penetration tube 66 can comprise a length 78 (e.g., measured along a direction that is substantially parallel relative to longitudinal axis 54) that is approximately any one of, or between approximately any two of, the following: 10, 20, 30, 40, 50, 60, 70, 80, 90, and 100 mm.

As shown, distal end 50 of body 18 includes an actuatable member 82. Actuatable member 82 includes a base 86 and a tip 90. In this embodiment, actuatable member 82 is movable relative to shaft 42 such that actuation of the actuatable member (e.g., by actuator 14) angularly displaces, by an angle 94, distal end 50 of the body relative to proximal end 46 of the body. More particularly, in the embodiment shown, actuatable member 82 is coupled to and distal to penetration tube 66, and tip 90 is coupled to and distal to base 86 of the actuatable member, such that actuation of the actuatable member angularly displaces the tip relative to the shaft. For example, actuatable member 82 is pivotally movable relative to shaft 42 about a pin 98 between a first position and a second position in which tip 90 is farther from longitudinal axis 54 of the shaft than when the actuatable member is in the first position. For example, actuatable member 82 includes a gear 99 that is concentric with pin 98 and whose movement is configured to be controlled by belt 30 of actuator 14 in order to move the actuatable member between the first position and the second position. Additional examples of suitable techniques for actuation and power transmission from motor 26 to actuatable member 82 are described in Examples section below.

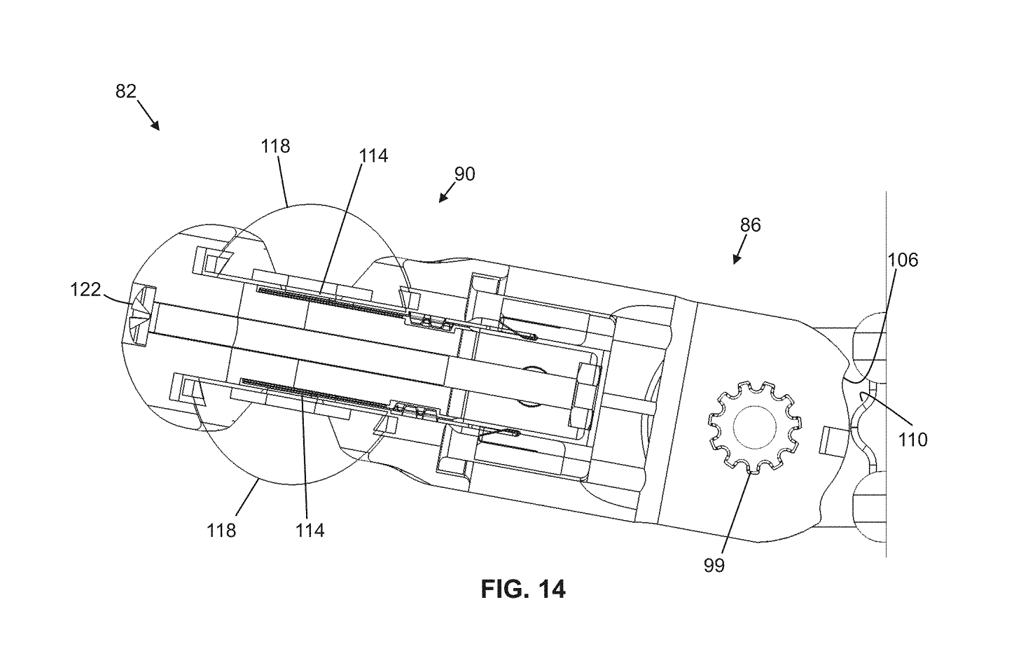

In this embodiment, in the second position, tip 90 may be displaced at any suitable angle 94, such as, for example, approximately any one of, or between approximately any two of, the following: 5, 10, 15, 20, 25, 30, 35, 40, 45, and 50 degrees. In this embodiment, angle 94 may be measured between a longitudinal axis 102 of actuatable member 82 and longitudinal axis 54 of shaft 42. For example, when actuatable member 82 is in the first position, longitudinal axis 54 of shaft 42 may be substantially aligned with longitudinal axis 102 of the actuatable member and, when the actuatable member is in the second position, the longitudinal axis of the actuatable member may be angularly disposed (e.g., at angle 94) relative to the longitudinal axis of the shaft. As shown in FIG. 14, as a safety feature, angular movement of actuatable member 82 relative to shaft 42 can be limited by contact between a stop surface 106 on the actuatable member and a stop surface 110 on penetration tube 66 of the shaft. Stop surfaces 106 and 110 can be spaced and/or angled relative to each other such that tip 90 of actuatable member 82 is limited to an angular displacement of approximately any one of, or between approximately any two of, the following: 30, 35, 40, 45, and 50 degrees.

Actuatable member 82 is movable from the first position to the second positon in any suitable duration of time, such as, for example, approximately any one of, or between approximately any two of the following: 0.25, 0.5, 0.75, 1, 1.5, 2, 2.5, and 3 seconds. Similarly, actuatable member 82 is movable from the second position to the first positon in any suitable duration of time, such as, for example, approximately any one of, or between approximately any two of the following: 0.25, 0.5, 0.75, 1, 1.5, 2, 2.5, and 3 seconds. Actuatable member 82 may be configured to be held in the second position for any suitable duration of time, such as, for example, approximately any one of, or between approximately any two of the following: 1, 2, 3, 4, 5, 6, 7, 8, 9, and 10 seconds. While actuatable member 82 moves between the first position and the second position, angle 94 may change linearly or non-linearly.

In this embodiment, actuatable member 82 is configured to angularly displace distal end 50 of body 18 relative to proximal end 46 substantially about a (e.g., single) axis (e.g., defined by pin 98). In other embodiments, however, an actuatable member (e.g., 82) may be configured to angularly displace a distal end (e.g., 50) of a body (e.g., 18) relative to a proximal end (e.g., 46) of the body about a first axis (e.g., defined by a pin (e.g., 98)) and about a second axis that is perpendicular to the first axis.

As shown, tip 90 comprises one or more sensors 114, each of which can be configured to collect data indicative of a force exerted on actuatable member 82. For example, in this embodiment, tip 90 includes one or more sensor covers 118, each of which can be configured to be disposed on a respective sensor 114 and transmit a force that is exerted on the sensor cover to the sensor. Each sensor cover 118 is configured to provide an interface between tissue and a respective sensor 114 such that tip 90 contacts tissue in a consistent and reproducible manner. For example, sensor 114 may be configured to collect data indicative of a force exerted on sensor cover 118 while the sensor cover contacts tissue. Sensor 114 of apparatus 10 may comprise any one or more suitable sensors configured to capture data indicative of force and/or pressure, such as, for example, a force-sensitive resistance sensor, a strain gauge sensor, a piezoelectric-based sensor (e.g., a piezoelectric-resistive, piezoelectric-capacitive, and/or the like sensor), a semiconductor-based sensor, a barometric sensor and/or the like.

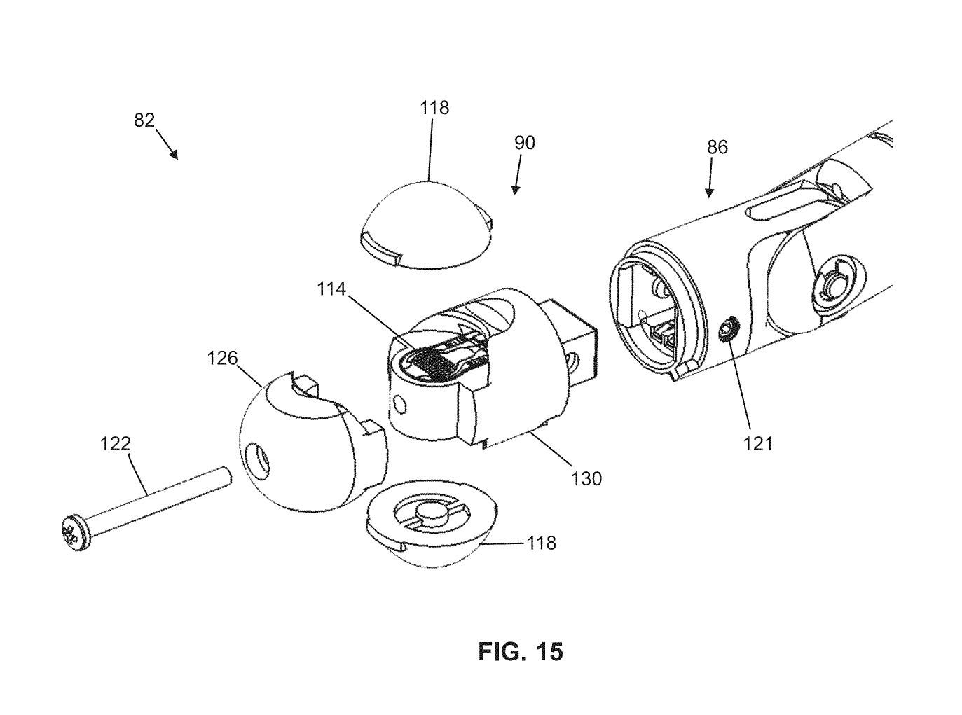

In this embodiment, tip 90 of actuatable member 82 may be configured to be removably coupled to base 86 of the actuatable member. For example, tip 90 may be releasably secured to base 86 via one or more fasteners 121. In this way and others, a tip (e.g., 90) of an actuatable member (e.g., 82) may be replaceable such that a variety of sensor(s) (e.g., 114) can be used with the actuatable member and/or such that the sensors are easily replaceable upon failure. As shown in FIGS. 14 and 15, each sensor 114 can be secured to tip 90 by sensor cover 118, which may be secured between a first member 126 of the tip and a second member 130 of the tip. For example, first member 126 and second member 130 of tip 90 can be secured to each other by a fastener 122. At least by securing sensor 114 in this way, sensor cover 118 may exert a preload on the sensor, which may provide more stable and/or consistent sensor outputs due to the preload. In some instances, one or more sensors 114 may require calibration before use. Additional examples of suitable techniques for preloading and calibrating sensors 114 are described in the Examples section below.

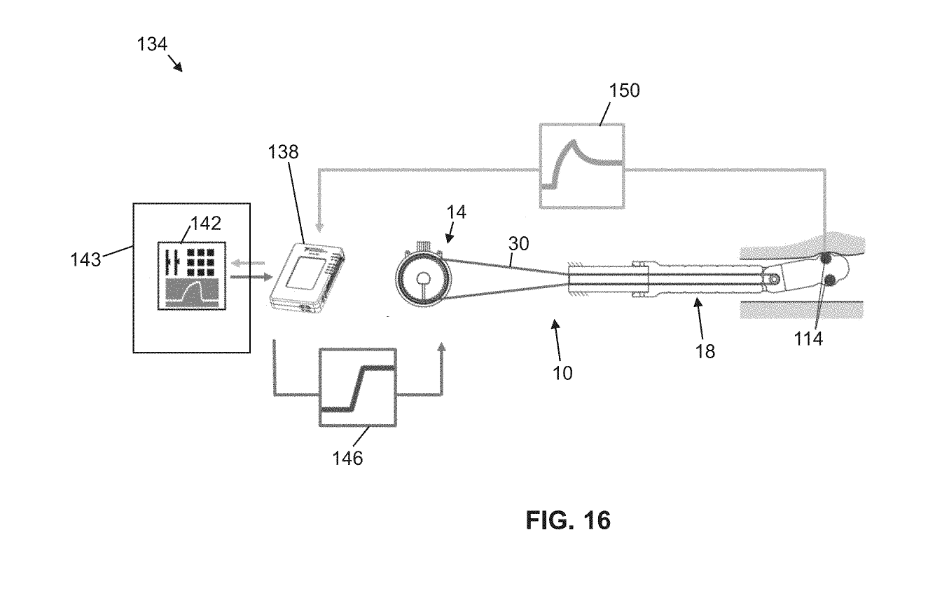

Referring now to FIG. 16, shown therein and designated by reference numeral 134 is an embodiment of the present systems for determining biomechanical (e.g., viscoelastic) properties of tissue. As shown, system 134 includes apparatus 10, a controller 138, and a graphics user interface (GUI) 142. GUI 142 may be included in a computing device 143 having a processor, memory, and/or storage. As shown, GUI 142 is configured to receive one or more test parameters (e.g., from a clinician) that define a desired actuation profile 146. In this embodiment, the test parameters comprises one or more of the following: a time duration of movement of actuatable member 82 from the first position to the second position, a time duration of holding the actuatable member in the second position, a time duration of movement of the actuatable member from the second position to the first position, an angle 94 of the actuatable member in the second position, and/or a linear or non-linear change in the angle from movement between the first and second positions. GUI 142 is configured to interface with a controller 138. Controller 138 controls actuator 14 in order to actuate actuatable member 82 based on desired actuation profile 146. Controller 138 can comprise a multifunction device having a data acquisition (DAQ) device that may be configured to receive a force profile 150 collected by sensor(s) 114 during movement between the first and second positions and transmit the force profile to computing device 143 (e.g., ultimately for display on GUI 142). In some embodiments, a computing device (e.g., 143) may comprise a DAQ configured to receive a force profile (e.g., 150) and display the force profile on a GUI (e.g., 142). GUI 142 can be configured to interface with DAQ of controller 138 and/or of computing device 143 such that the GUI displays (e.g., in real-time) force profile 150 collected by sensor(s) 114. For example, computing device 143 may apply a mathematical model (e.g., a Maxwell model, a Kelvin-Voight model, a standard linear solid (SLS) model, a quasilinear viscoelastic (QLV) model, and/or the like) to force profile 150 to determine one or more coefficients of the model, which may quantitatively indicate the viscoelastic properties of the tissue being examined. Computing device 143 may be configured to display such coefficients on GUI 142. Additional examples of mathematical modeling to determine viscoelastic properties of tissue are described in the Examples section below. Further, additional examples of determining biomechanical (e.g., viscoelastic) properties of vaginal tissue in six patients are described in the Examples section below.

### EXAMPLES

The present invention will be described in greater detail by way of specific examples. The following examples are offered for illustrative purposes only and are not intended to limit the invention in any manner. Those of skill in the art will readily recognize a variety of noncritical parameters that can be changed or modified to yield essentially the same results.

### Example 1

### Force Sensor

In this example, the sensor (e.g., 114) tested was the Interlink Electronics FSR 400 Short Force-sensitive Resistor, available from Interlink Electronics, Inc., of Westlake Village, Calif., USA. The sensor (e.g., 114) included a polymer thick-film device that contained a conductive pattern. When the sensor (e.g., 114) was compressed, the resistance of the conductive pattern would decrease relative to the compression force applied to it.

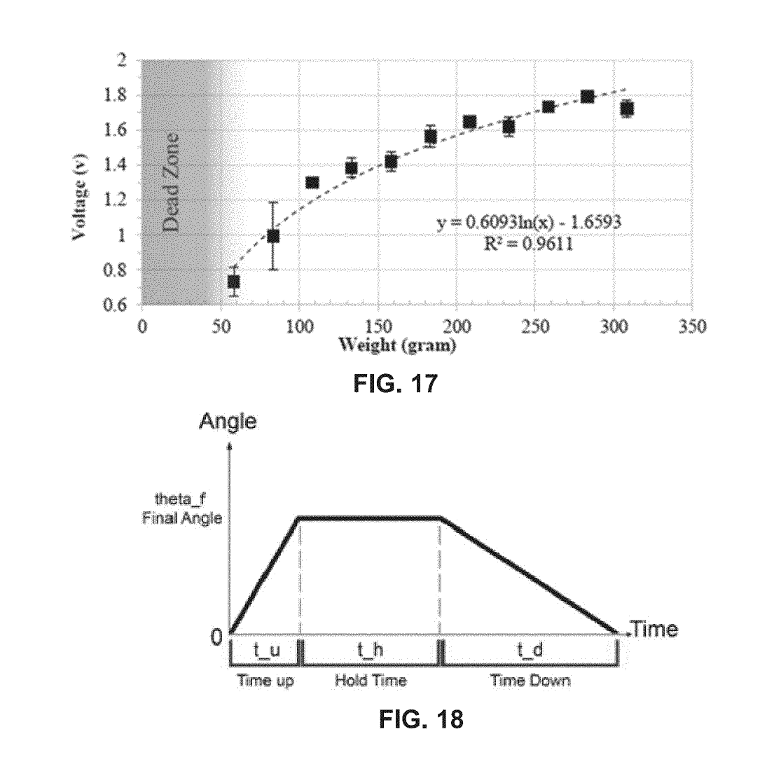

The sensor (e.g., 114) required at least 50 gr of equivalent force before generating a stable sensor output, which required the user to preload the sensor before using it for measurements below that threshold; otherwise, the user risked operating in the “dead zone” where no force is being sensed. FIG. 17 depicts the averaged calibration data points and calibration curve (dotted line).

The repeatability of the response of the sensor (e.g., 114) was highly dependent on the repeatability of the sensor engagement method. It was observed that the sensitivity of the sensor (e.g., 114) increased if the engagement surface pressing on the active area is smaller than the active area, rather than larger or equal to it. Thus, the engagement surface was designed to be 3.8 mm in diameter. It was also observed that changes in the positioning or hardness of the sensor engagement surface causes changes in the sensor sensitivity. Therefore, it was determined that a repeatable actuation method was needed to produce repeatable force measurements.

A. Sensor Engagement and Preload

Since the repeatability of the sensor (e.g., 114) was highly affected by the engagement method/surface, a loading mechanism was designed to load the sensor in a consistent manner. Additionally, the loading mechanism is also used to preload the sensor (e.g., 114), due to the existence of the “dead zone” between 0 and 50 gr where the sensor output is either unstable or non-existent (see FIG. 17). The loading mechanism consisted of a hemisphere for tissue contact with a pressure button, housed inside the 2-part fingertip (e.g., 90). The bolt (e.g., 122) presses the two parts (e.g., 126, 130) of the fingertip together, which in turn presses on the hemisphere (e.g., 118), which in turn presses on the sensor (e.g., 114) to preload it (see e.g., FIG. 14).

It was observed that the output voltage response between 100 gr and 300 gr applied equivalent force can be assumed to be linear, which makes it the optimal range of operation for the sensor (e.g., 114). Therefore, a preload exceeding 100 gr was preferred.

B. Sensor Characterization and Calibration

The sensor output curves provided by the manufacturer were for demonstration purposes only due to the large variability in the response depending on the working conditions. Per the manufacturer integration guidelines, it was recommended to calibrate the sensor in its working environment. Hence, a calibration rig was designed to perform a static force calibration on the sensor (e.g., 114) using dead weights. The rig consisted of a vertical carriage that holds dead weights, and was fitted with a pressure plunger that presses on the tissue contact hemisphere (e.g., 118). The fingertip (e.g., 90) is attached onto the rig in the same way it is attached on body (e.g., 18).

The calibration procedure was setup as follows: The sensor (e.g., 114) was tested using dead weights ranging from 25 gr to 300 gr and discretized in steps of 25 gr. This translated into 12 different weight steps. Each weight step consisted of loading the carriage with the required weight and slowly lowering it to press on the skin contact surface (e.g., 118) of the fingertip. Once it pressed, a DAQ device (e.g., 138) was used to record the voltage V′ₒᵤₜ for 30 seconds to allow the signal to settle. Then, the carriage was lifted and prepared for the next weight step. The time between each two steps was enough to allow the sensor (e.g., 114) to recover and release any residual stress.

The steps were not incrementally tested (as in 25 gr, 50 gr, 75 gr, etc.), but rather randomized using an online randomizer in order to prevent any biasing in the response results. Overall, three runs of the complete 12 steps were performed, with the testing sequence randomized before each run.

It was observed that at an applied weight of 25 gr, the sensor (e.g., 114) produced either no signal or an unstable signal. Therefore, the data set for an applied weight of 25 gr was discarded. The sensor (e.g., 114) response to the step load appeared to match a first-order system response, with the voltage rising logarithmically and settling at a specific value after a certain time period. Therefore, to characterize the time domain characteristics of the sensor (e.g., 114), the test data for each weight step was averaged and curve-fit on a first-order response.

After testing was completed, the data was compiled and analyzed, see FIG. 17, to produce the calibration equation. This equation is valid for an applied voltage V₋+=5V, R_{M}=3 KΩ, and weights between 50 to 300 gr.

The maximum deviation from the average reading was 15% and occurred on 75 gr, which was likely due to the sensor (e.g., 114) sustaining some impact forces from the dead weight. The time constant of the sensor response, τ, is defined as the time needed for the output voltage to reach 63.2% of its steady-state value. Assuming that the sensor steady-state value is the final value at t=30 s, the average time constant of the sensor response was approximately 0.21 s. This corresponds to a settling time Ts=4τ=0.84 s.

Some important conclusions were drawn based on the calibration results. First, for the purposes of the system, the loading time of the vaginal tissue was expected to be between 0.5 and 3 seconds. A 0.84 s settling time is somewhat higher than desired since the individual data points during the tissue loading period are used in the characterization procedure of the vaginal tissue. It is also important to note that at the applied weight of 25 gr, the sensor (e.g., 114) produced either no signal or an unstable signal; this showed that the sensor possesses a “dead zone” for loads less than approximately 50 gr. Another important realization from the calibration results was that the response of the sensor (e.g., 114) can be assumed to be linear between 100 gr and 300 gr.

Building on these findings, calibration was redone with an induced preload of 75 gr on the sensor (e.g., 114) to surpass the “dead zone” between zero and 50 gr. The calibration procedure with the preload was different than the initial calibration with no preload on the sensor. The applied loads were not randomly-ordered, but rather increased and then decreased incrementally. The reason for this calibration procedure change was to attempt to recreate the increasing force on the sensor as it presses on the soft tissue then the decreasing force as the tissue relaxes. It was found that each time the load was removed, the sensor baseline voltage increased rather than remaining constant. Additionally, between each two identical load steps (loading and unloading), there was an increase in the reading of up to 10% of full-scale. It was concluded that the sensor retains some of the applied load due to its inability to fully recover.

The sensor characterization revealed the limitations and weaknesses of the FSR sensor, which were not clearly apparent at the time of purchase. Without any preload on the sensor, a “dead zone” exists between 0 and 50 gr where no load is read; additionally, the sensor settling time was found to be within the range of the expected loading time (T_{settling}=0.84 s compared to T_{loading}=0.5 s to 3 s). With preload on the sensor, the sensor was unable to recover when unloaded and the sensor readings will become inaccurate with usage. It was concluded that the FSR sensor may sufficiently work as a proof-of-concept, however a more specialized and more reliable sensor (e.g. load cell, piezoresistive, etc.) could be used for measuring the tissue reaction forces.

### Actuator and Power Transmission

In this example, for actuation and power transmission, a DC-motor (e.g., 26) was used along with a timing belt for power transmission. Initially, a servo motor was used to actuate the finger. However, the servo motor suffered from a few important limitations. First, the servo motor had repeatability issues under load, with the position error of the motor being about ±8 degrees. Second, since the servo control circuit was embedded within the motor, a feedback signal to verify the current position could not be acquired. These limitations were addressed by using a high-end DC-motor/encoder combination. The DC-motor was driven using pulse width modulation (PWM) to position within the needed motor angle, and the encoder feedback was read to control the position. In this example, the motor (e.g., 26) used was the Faulhaber 3557CS with a gearbox and encoder. Its high stall torque (1.6 Nm) and high no-load speed (128 rpm) were well above the working requirements of the system.

The angular positioning of the motor was controlled using PID control. The PID gains were selected to prevent overshoot and minimize steady state error. The desired motor angle was set by the user, while the current motor angle was read by the attached encoder. The error between the desired and the current angle was fed into the PID controller, and the controller output was converted into a PWM signal that was used to drive the motor. For power transmission to the body (e.g., 18), a timing belt was used with a 1:4 ratio (1 motor rotation=4 rotations of actuatable member (e.g., 82)).

The body (e.g., 18) applied a given displacement profile on the soft tissue via the actuatable member (e.g., 82). This displacement profile corresponded to a certain actuation profile (e.g., 146) for the motor (e.g., 26). The actuation profile (e.g., 146) of the motor (e.g., 146) consisted of three actions, shown in FIG. 18: ramp upward (tissue loading) for a time duration tᵤ between 0.5 s and 3 s, hold (tissue relaxation) for a time duration tₕ between 1 s and 10 s, ramp downward (tissue unloading) for a time duration t_{d} between 0.5 s and 3 s.

The actuation profile (e.g., 146) was parametrized by the different durations (tᵤ, tₕ, t_{d}) and the final angle θ_{f}. The administering physician was able to define these parameters as desired using the Graphical User interface (GUI) (e.g., 142). During tᵤ, the tissue was being indented by the actuatable member (e.g., 82); during tₕ the tissue was being held at a fixed strain level; during t_{d} the tissue is being released by retracting the actuatable member.

### Actuator and Power Transmission

The literature survey conducted on viscoelastic modeling of human soft tissue showed that biological soft tissue behaves as a non-linear viscoelastic solid. A viscoelastic material is one that exhibits stress relaxation, creep, and hysteresis, and viscous behavior causes a time-dependent response in the material upon loading. Linearity is concerned with the dependence or independence of the response on the response time history. In other words, if the material properties change as the material is subjected to a certain stress or strain, the material is considered non-linear.

A literature survey of more than 25 papers concluded that there are 2 main approaches for modeling viscoelastic behavior of soft tissue. First, the differential model which uses analogous mechanical elements such as springs and dashpots, and results in a model equation that is typically a differential equation of first or second order. Examples include the Maxwell model, the Kelvin-Voight model, and the Standard Linear Solid (SLS) model. The second model is the integral model which uses integro-differential equation to model the viscoelastic behavior. For example, the Boltzmann Superposition Model and Fung's Quasilinear Viscoelastic Model are integro-differential models.

The literature survey also showed that there is no strong consensus around a preferred model for soft tissue modeling. Studies were often conducted with the sole purpose of designing more accurate models of soft tissue behavior. Therefore, the examples outlined in this document used the most basic models as a starting point for comparison with the test data acquired from the patients.

A. Maxwell Model

The Maxwell model is one of the simplest viscoelastic model that can be used to curve-fit a force response due to a given displacement (or indentation). The Maxwell model is a linear differential model that uses a spring and dashpot in series to include both the elastic and viscous portions of the response.

When the model is subjected to a certain force f, both the spring and dashpot produce a corresponding reaction force. The reaction force of the spring is directly proportional to the displacement x, while that of the dashpot is proportional to the displacement rate dx/dt. The proportionality factors of the spring and dashpot are called the spring constant, k, and the damping coefficient, b, respectively, which are assumed to be fixed in this example.

The force relaxation response of a Maxwell model is characterized by the complete dissipation of the force—where the force due to a step displacement goes to zero at final time. The characteristic parameter for a 1st order system, such as the Maxwell model, is the time constant τ=b/k. This parameter can be used to characterize the behavior of the vaginal tissue by curve-fitting the force response to Equations 11 and 13.

Because both the spring and dashpot were subjected to the same force f (t), then:

$$\begin{matrix}f ( t ) = k [ {x}_{2} ( t ) - {x}_{3} ( t ) ] & Equation 5 \\ f ( t ) = b \frac{d}{dt} [ {x}_{1} ( t ) - {x}_{2} ( t ) ] & Equation 6\end{matrix}$$

Because the total deformation u(t)=x₁−x₃=(x₁−x₂)+(x₂−x₃), then

$$\begin{matrix}\frac{d}{dt} u ( t ) = \frac{d}{dt} ( {x}_{1} - {x}_{2} ) + \frac{d}{dt} ( {x}_{2} - {x}_{3} ) & Equation 7 \\ \frac{d}{dt} u ( t ) = \frac{1}{k} \frac{d}{dt} f ( t ) + \frac{1}{b} f ( t ) & Equation 8 \\ ( \frac{b}{k} D + 1 ) f = b \cdot Du & Equation 9\end{matrix}$$

Then, the force response is a first order differential equation with the displacement (t) as an input or a forcing function. Consequently, the actual force response is both dependent on time and on the input displacement. The response for a ramp input or a step input could be predicted as follows.

For a step input (t)=u₀, Equation 9 becomes

$$( \frac{k}{b} D + 1 ) f = 0 \qquad Equation 10$$

Then, solving the differential equation by assuming zero initial conditions, the force response is in the form:

$$f ( t ) = {ku}_{o} exp ( - \frac{k}{b} t ) \qquad Equation 11$$

For a ramp input (t)=vt, Equation 6 becomes:

$$( \frac{k}{b} D + 1 ) f = bv \qquad Equation 12$$

Then, solving the differential equation by assuming zero initial conditions, the force response is in the form:

$$f ( t ) = bv [ 1 - exp ( - \frac{k}{b} t ) ] \qquad Equation 13$$

The force relaxation response of a Maxwell model is characterized by the complete dissipation of the force—where the force due to a step displacement goes to zero at final time. From Equations 11 and 13, the characteristic parameter for a 1st order system, such as the Maxwell model, is the time constant τ=b/k. This parameter can be used to characterize the behavior of the vaginal tissue by curve-fitting the force response to Equations 11 and 13.

B. Standard Linear Solid (SLS) Model

A better suited model for this example was the Standard Linear Solid (SLS) model. Whereas the Maxwell model is unable to model creep behavior, the SLS model can be used to model both creep and stress relaxation behavior for many viscoelastic materials. Additionally, the SLS model force relaxation response is not characterized by complete dissipation of the force. Some residual amount of force was conserved in the system at the final time. Therefore, as the name suggests, the Standard Linear Solid model was more suitable for modeling linear viscoelastic solids.

The SLS model consists of a Maxwell model (spring and dashpot in series) parallel with a spring. The spring rate of the series spring is kₛ while that of the parallel spring is kₚ, and the damping coefficient of the dashpot is b, which were assumed to be fixed in this example.

In this example, (t)=x₃−x₁ was defined as the total displacement acting on the model, uₛ=x₃−x₂ was defined as the displacement acting on the Maxwell spring, and u_{d}=x₂−x₁ was defined as the displacement acting on the Maxwell damper. Therefore,

u(t)=uₛ(t)+u_{d}(t) Equation 14

From the previous subsection, the two components of the Maxwell model were subjected to the same force, fₛ=f_{d}

$${k}_{s} {u}_{s} ( t ) = b \frac{d}{dt} {u}_{d} ( t ) \qquad Equation 15$$

Rearranging Equation 15 yields,

$$\frac{d}{dt} {u}_{d} ( t ) = \frac{{k}_{s}}{b} {u}_{s} ( t ) \qquad Equation 16$$

The total force acting on the system was equal to the sum of the forces acting on each leg; where the force acting on the parallel spring is fₚ and the force acting on the Maxwell leg is fₛ

f(t)=fₚ+fₛ=kₚu(t)+kₛuₛ(t) Equation 17

Rearranging Equation 17 yields:

$${u}_{s} ( t ) = \frac{1}{{k}_{s}} f ( t ) - \frac{{k}_{p}}{{k}_{s}} u ( t ) \qquad Equation 18$$

Taking the time derivative of Equation 14 and substituting Equations 16 and 18,

$$\frac{d}{dt} u ( t ) = \frac{d}{dt} {u}_{s} ( t ) + \frac{d}{dt} {u}_{d} ( t ) \\ \frac{d}{dt} u ( t ) = \frac{1}{{k}_{s}} \frac{d}{dt} f ( t ) - \frac{{k}_{p}}{{k}_{s}} \frac{d}{dt} u ( t ) + \frac{1}{b} f ( t ) - \frac{{k}_{p}}{b} u ( t ) \qquad Equation 19$$

Rearranging Equation 19 yields:

$$( \frac{b}{{k}_{s}} D + 1 ) f ( t ) = [ ( b + \frac{{bk}_{p}}{{k}_{s}} ) D + {k}_{p} ] u ( t ) \\ ( {τ}_{f} D + 1 ) f ( t ) = ( {τ}_{u} D + 1 ) {k}_{p} u ( t ) \qquad Equation 20$$

where

$${τ}_{f} = \frac{b}{{k}_{s}} and {τ}_{u} = \frac{b}{{k}_{s}} + \frac{k}{{k}_{p}} .$$

Similar to the Maxwell model, the force response of the SLS model, Equation 20, can be described as a first order linear differential equation with the displacement (t) as an input or a forcing function. The actual force response is both dependent on time and on the input displacement.

For a step input (t)=u₀, solving the differential equation with zero initial conditions, the force response is:

$$f ( t ) = {k}_{p} {u}_{o} [ 1 - ( 1 - \frac{{τ}_{u}}{{τ}_{f}} ) exp ( - \frac{t}{{τ}_{f}} ) ] \qquad Equation 21$$

For a ramp input u(t)=vt, solving the differential equation with zero initial conditions, the force response is:

$$f ( t ) = {k}_{p} v [ t + ( {τ}_{u} - {τ}_{f} ) ( 1 - exp ( - \frac{t}{{τ}_{f}} ) ) ] \qquad Equation 22$$

The force relaxation response of the SLS model is characterized by a partial dissipation of the force, which is more suitable for a viscoelastic solid. Similar to the Maxwell model, it is apparent from Equations 21 and 22 that the characteristic parameter is the time constant τ_{f}=b/k associated with the exponential decay. Therefore, whichever of the two models is to be used, the exponential time constant can be used to characterize the response of the system.

C. Quasilinear Viscoelastic (QLV) Model

Another model used to model soft tissue behavior and presented in this example is an integro-differential model called the Quasilinear Viscoelastic (QLV) Model. The QLV model is given by the following equation:

$$σ ( t ) = {\int }_{0}^{t} \{ K ( t - τ ) \frac{d}{d τ} [ {σ}^{e} ( λ ( τ ) ) ] \} d τ \qquad Equation 23$$

where σ is the stress, λ is the stretch, σ^{e} is the elastic response, and K(t) is the reduced relaxation function. Fung proposed K(t) as:

$$K ( t ) = \frac{1 + c {\int }_{{τ}_{1}}^{{τ}_{2}} \frac{1}{τ} exp ( - \frac{t}{τ} ) d τ}{1 + c ln ( \frac{{τ}_{2}}{{τ}_{1}} )} \qquad Equation 24$$

Despite the QLV model being more difficult to implement than most differential models, literature suggest that the QLV model produces more accurate results and accounts for the non-linear behavior of soft tissue over a continuous spectrum of stress relaxation times. Therefore, the QLV model may be useful to predict the non-linear behavior of soft tissue.

### Data Analysis

The system (e.g., 134) was used by a physician to perform as set of measurements on six patients which were given the following identifiers to conceal their true identity: DR, JS, CS, KD, RC, and RG. Out of six patients, two (RC and RG) had some degree of prolapse while the other four had no prolapse. The tests were conducted using three different angle schemes of 10, 15, and 20 degrees of rotation of the actuatable member (e.g., 82). Additionally, they were conducted using two different timing schemes of 1-1-1 and 1-6-1, where the numbers translate to indentation-hold-release times (measured in seconds). There were 6 tests per patient, adding up to 36 tests. The system was also programmed to repeat each test three times to account for any abnormalities. The research aims to explore the feasibility of using the loading data during the indentation period to obtain a characteristic measure of the tissue viscoelastic behavior.

Some individual tests exhibited the symptoms predicted from the sensor characterization, where the sensor retained some of the applied load due to the preload. For the same test parameters, it was clear that the sensor baseline and reading were rising as the test was repeated. For example, the average standard deviation of the test was 0.011V or 16% of the test range. While not all tests have exhibited this issue, its presence reduced the confidence in the test results, and highlighted the need for a more repeatable sensor. Nonetheless, some of the tests were more repeatable. For example, the average standard deviation of the test conducted on JS was 0.005V or 4.6% of the test range.

Comparing the different tests, it was observed that the sensor voltage output in the hold period increases as the indentation angle increases as expected. However, it was also noted that the sensor baseline often differed between tests for the same patient. This observation again raised questions whether any conditions changed between tests (the device was moved, the patient shifted, etc.), or whether the sensor retained any load, or both. This uncertainty in the sensor data prevents an accurate comparison between test results.

For the purpose of conducting the analysis, it was possible to disregard the sensor errors, and assume that the shift in baseline is only due to a difference between testing conditions. Then, the curves can be normalized by subtracting the lowest baseline value from each data point. This normalization effectively reduced the baseline of all the curves to zero. Furthermore, rather than comparing the maximum voltage value, a more accurate measure of the difference in tissue behavior between the curves/tests is the time constant τ.

The time constant is a measure of the viscoelastic behavior of the tissue. A high time constant indicates high damping effects in the tissue, and as such the tissue takes longer time to respond to the force and vice-versa. A linear viscoelastic material should have the same time constant regardless of the strain rate or stress-strain history in general, considering that all testing conditions remain the same. On the other hand, a non-linear viscoelastic material would exhibit a change in the response time between different strain histories.

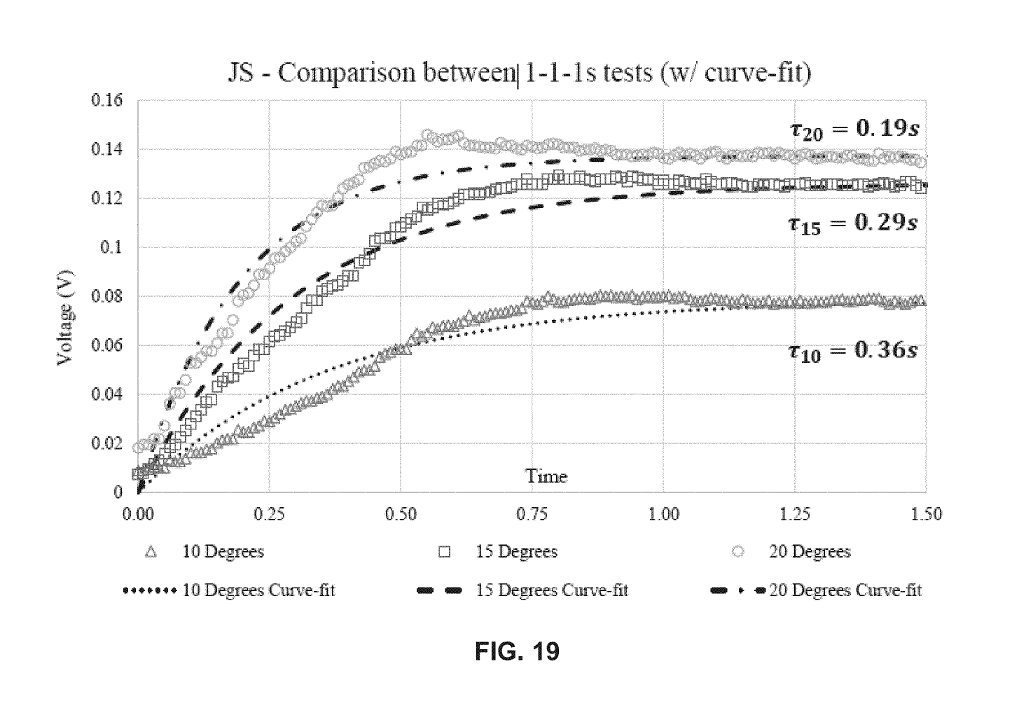

The data was curve-fit using the Maxwell model. The curve-fit was based on minimizing the Root Mean Square Error (RMSE) between the model curve and the experimental data which can be seen in FIG. 19. Two important observations were made. First, the Maxwell model is effective to a certain extent in completing the curve-fitting task accurately. For instance, the RMSEs for the 10, 15 and 20 degree curve-fits are 0.005V, 0.006V and 0.006V respectively. This corresponded to 6.9%, 5.1% and 4.6% of the final value. Second, based on the curve-fits using the Maxwell model, the time constant differed between different strain rates for the same patient.

This result was expected due to the fact that soft tissue is a non-linear viscoelastic material while the Maxwell model is a linear model. This latter observation also alludes to the fact that a non-linear viscoelastic model may be used in other embodiments of the system.

Finally, a shortcoming of the sensor which was overserved in the test data is the fact that the sensor load retention may be causing the loss of the force relaxation profile. During the hold period of the test, the tissue was expected to relax and the reaction force to slowly decrease from the value reached after indentation. Conversely, the sensor signal was found to be rising slightly as it settles. This can be explained by the sensor retaining some of the applied load and continuing to settle during the hold period. It may be beneficial with future tests to investigate whether this trend will persist during longer hold times such as 20 or 30 seconds.

In summary, the in-vivo test data proved the feasibility of the system in obtaining force-time data from patients given a specific indentation profile, as well as the feasibility of using this data to characterize the viscoelastic behavior of the tissue. This characterization can later be correlated to certain factors or conditions, such as age, childbirth, or others, that may adversely affect the soft tissue.

### Example 2

In this example, the objective was to compare reaction forces of the human anterior vaginal wall in control (C) and prolapsed (P) women in response to pressure applied at different angles of indentation through an automated system (e.g., 134) having a distal sensor (e.g., 114).

A. Method

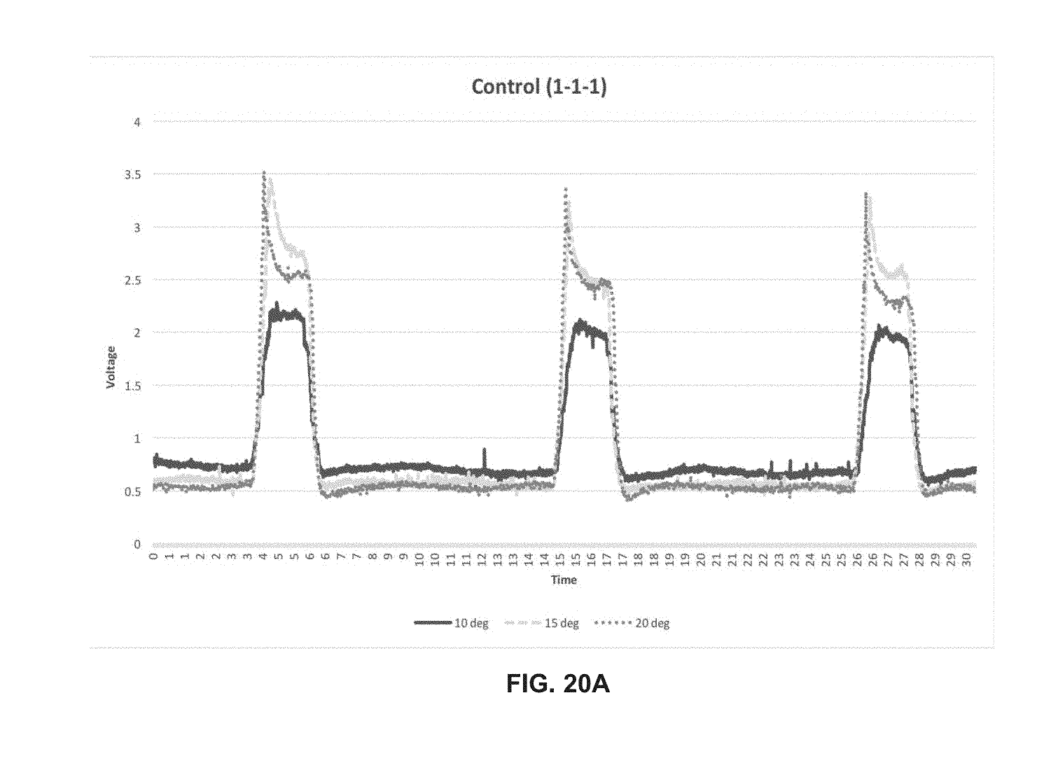

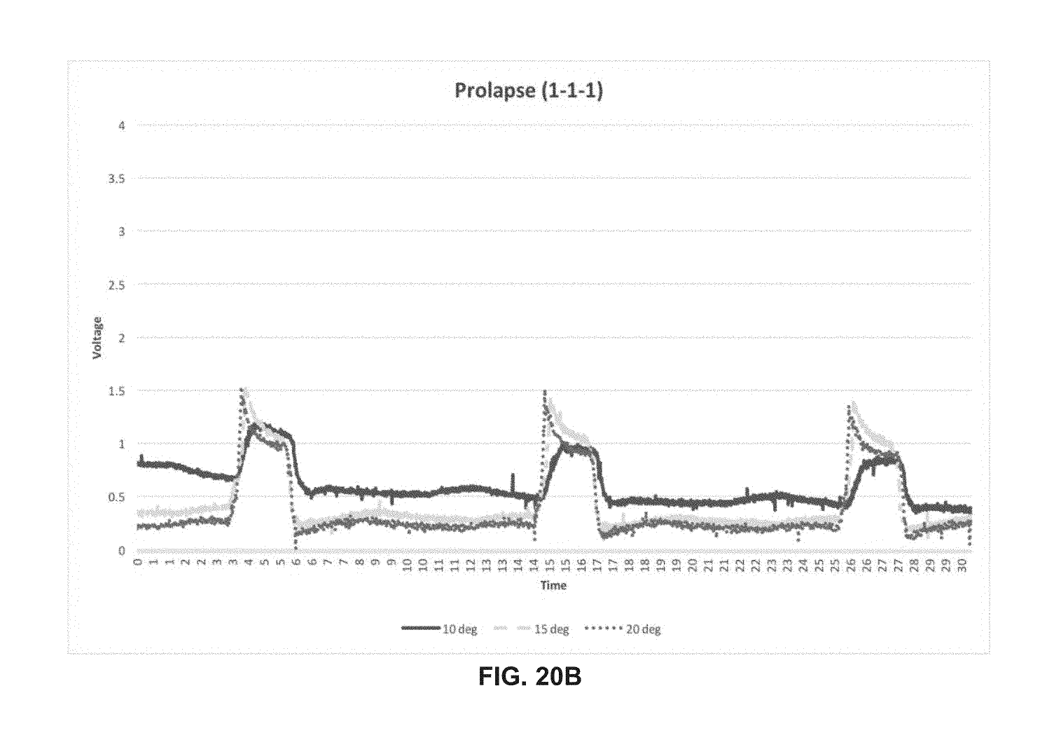

In this example, a tripod-mounted, body (e.g., 18) equipped with a calibrated, polymer thin-film sensor (e.g., 114) at its tip (e.g., 90) and automated by NI LabView 2105 software for motion control via an actuator (e.g., 14) was used to create anterior vaginal wall deformations at 10, 15 and 20 degree angles. Age-matched women in the C and P groups were compared. All measurements were performed in the supine position in the operating room, with patients under general anesthesia prior to the start of the operation and after the bladder was drained. Each deformation included a 1 second upwards indentation, a 1 second maintenance “hold”, and a 1 second return of the fingertip to the baseline. Measurements were done in triplicate with a three second interval between each deformation sequence. Real-time voltages, equivalent to reaction forces measured by the sensor during each indentation, were modeled as motion profile curves and analyzed (see FIGS. 20A and 20B). The motion profile curve of each indentation was used to calculate baseline voltage, amplitude change over the one second interval of upwards indentation, and slope of the upwards indentation curve in the median 0.5 second range (see Table 1 below).

TABLE 1
Mean voltage values of triplicate indentation
profiles at 10°, 15°, 20°

| | |
|---|---|
| Control (n = 5) | Prolapse (n = 5) |

10 degrees

| | | |
|---|---|---|
| Baseline | 0.94 ± 0.24 | 0.58 ± 0.32 |
| Amplitude change | 1.0 ± 0.43 | 0.53 ± 0.3 |
| Indentation slope | 0.85 ± 0.7 | 0.77 ± 1.35 |

15 degrees

| | | |
|---|---|---|
| Baseline | 0.75 ± 0.27 | 0.38 ± 0.24 |
| Amplitude change | 0.92 ± 0.55 | 1.11 ± 0.42 |
| Indentation slope | 0.62 ± 0.56 | 1.14 ± 0.58 |

20 degrees

| | | |
|---|---|---|
| Baseline | 0.67 ± 0.3 | 0.32 ± 0.23 |
| Amplitude change | 1.73 ± 0.84 | 1.00 ± 0.53 |
| Indentation slope | 1.9 ± 1.52 | 1.56 ± 1.51 |

B. Results

Five women of similar age group (mean 64, 51-73) were studied in each group. A significant difference was observed between all degrees of indentation in baseline voltage in P and C groups (p<0.05). At 10 and 20 degrees of indentation, there was a significant difference in amplitude change between P and C groups, while there was a significant difference in slope of indentation at 15 degrees between P and C groups.

C. Conclusions

The biomechanical properties of the human anterior vaginal wall can be objectively determined by the present system (e.g., 134). The system can apply a predictable and reproducible deformation to the anterior vaginal wall to compare the indentation properties of vaginal tissue in prolapsed and non-prolapsed conditions.

Some embodiments of the present methods for determining biomechanical properties of tissue comprise inserting a distal end (e.g., 50) of an apparatus (e.g., 10) into an insertion site (e.g., a vagina) on a patient, wherein the apparatus comprises: an elongated body (e.g., 18) having a proximal end (e.g., 46) and a distal end (e.g., 50), the elongated body including: a shaft (e.g., 42) having a longitudinal axis (e.g., 54); and an actuatable member (e.g., 82) pivotally coupled to the shaft, wherein: the actuatable member comprises a sensor (e.g., 11) that is configured to collect data indicative of a force exerted on the actuatable member, the actuatable member is movable relative to the shaft between a first position and a second position in which a tip (e.g., 90) of the actuatable member is farther from the longitudinal axis of the shaft than when the actuatable member is in the first position; actuating the actuatable member between the first position to the second position; sensing, via the sensor, data indicative of the force exerted on the actuatable member while actuating the actuatable member between the first position and the second position.

Some embodiments of the present methods include receiving one or more test parameters that define a desired actuation profile (e.g., 146). Some embodiments of the present methods include actuating the actuatable member between the first position and the second position according to the desired actuation profile. Some embodiments of the present methods include curve-fitting a viscoelastic model to the data sensed by the sensor.

Some embodiments of the present methods include holding the actuatable member in the second position for approximately one second to approximately ten seconds. In some embodiments, the data indicative of the force exerted on the actuatable member is sensed while holding the actuatable member in the second position. In some embodiments, the actuatable member is actuated from the first position to the second position in approximately one-half seconds to approximately three seconds. In some embodiments, the actuatable member is actuated from the second position to the first position in approximately one-half seconds to approximately three seconds.

The above specification and examples provide a complete description of the structure and use of illustrative embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the methods and systems are not intended to be limited to the particular forms disclosed. Rather, they include all modifications and alternatives falling within the scope of the claims, and embodiments other than the one shown may include some or all of the features of the depicted embodiment. For example, elements may be omitted or combined as a unitary structure, and/or connections may be substituted. Further, where appropriate, aspects of any of the examples described above may be combined with aspects of any of the other examples described to form further examples having comparable or different properties and/or functions, and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1. An apparatus for determining biomechanical properties of tissue, the apparatus comprising:
an elongated body having a proximal end and a distal end, the elongated body including:
a shaft having a longitudinal axis; and
an actuatable member pivotally coupled to the shaft, the actuatable member comprising:
a base; and
a tip configured to be coupled to the base, the tip having a sensor that is configured to collect data indicative of a force exerted on the actuatable member;
wherein the actuatable member is movable relative to the shaft between a first position and a second position in which the tip is farther from the longitudinal axis of the shaft than when the actuatable member is in the first position.

2. The apparatus of claim 1, wherein the shaft includes a stop configured to limit pivotal movement of the actuatable member relative to the shaft.

3. The apparatus of claim 2, wherein the stop limits pivotal movement of the actuatable member to approximately 50 degrees from the first position.

4. The apparatus of claim 1, wherein the tip is configured to be detachably coupled to the base.

5. The apparatus of claim 1, wherein the tip comprises a sensor cover configured to transfer the force exerted on the actuatable member to the sensor.

6. The apparatus of claim 1, wherein the body includes gradations configured to indicate a distance relative to the sensor.

7. The apparatus of claim 1, comprising an actuator configured to move the actuatable member between the first position and the second position.

8. The apparatus of claim 7, wherein the actuator comprises a motor coupled to the actuatable member via a belt such that the motor moves the actuatable member by moving the belt.

9. A system for determining biomechanical properties of tissue, the system comprising:
an apparatus coupled to the frame, wherein the apparatus includes:
an elongated body having a proximal end and a distal end, the elongated body including:
a shaft having a longitudinal axis; and
an actuatable member pivotally coupled to the shaft, the actuatable member comprising:
a base; and
a tip configured to be coupled to the base, the tip having a sensor that is configured to collect data indicative of a force exerted on the actuatable member;
wherein the actuatable member is movable relative to the shaft between a first position and a second position in which the tip is farther from the longitudinal axis of the shaft than when the actuatable member is in the first position;
an actuator, the actuator configured to move the actuatable member between the first position and the second position;
a graphics user interface (GUI) configured to receive one or more test parameters; and
a controller configured to control the actuator based on the one or more test parameters and receive, from the sensor, data indicative of the force exerted on the actuatable member.

10. The system of claim 9, wherein the one or more test parameters comprise one or both of the following:
a time duration of movement of the actuatable member from the first position to the second position;
a time duration of holding the actuatable member in the second position; and
a time duration of movement of the actuatable member from the second position to the first position.

11. The system of claim 9, wherein the tip is configured to be detachably coupled to the base.

12. A method for determining biomechanical properties of tissue, the method comprising:
inserting a distal end of an apparatus into an insertion site on a patient, wherein the apparatus comprises:
an elongated body having a proximal end and a distal end, the elongated body including:
a shaft having a longitudinal axis; and
an actuatable member pivotally coupled to the shaft, wherein:
the actuatable member comprises a sensor that is configured to collect data indicative of a force exerted on the actuatable member,
the actuatable member is movable relative to the shaft between a first position and a second position in which a tip of the actuatable member is farther from the longitudinal axis of the shaft than when the actuatable member is in the first position;
actuating the actuatable member between the first position to the second position;
sensing, via the sensor, data indicative of the force exerted on the actuatable member while actuating the actuatable member between the first position and the second position.

13. The method of claim 12, comprising holding the actuatable member in the second position for approximately one second to approximately ten seconds.

14. The method of claim 13, wherein the data indicative of the force exerted on the actuatable member is sensed while holding the actuatable member in the second position.

15. The method of claim 12, wherein the actuatable member is actuated from the first position to the second position in approximately one-half seconds to approximately three seconds.

16. The method of claim 12, wherein the actuatable member is actuated from the second position to the first position in approximately one-half seconds to approximately three seconds.

17. The method of claim 12, comprising curve-fitting a viscoelastic model to the data sensed by the sensor.
